(19) United States
(12) Patent Application Publication
(10) Pub. No.: US 2025/0127698 A1
(43) Pub. Date: Apr. 24, 2025

(54) Hair Perming Compositions With Amine Derivatives

(71) Applicant: ELC Management LLC, Melville, NY (US)

(72) Inventors: Xiuhong Zhai, Vadnais Heights, MN (US); Jeanna Zguris, North Oaks, MN (US); Chad Pavlis, Lino Lakes, MN (US); Tanushree Tokle, Lino Lakes, MN (US)

(21) Appl. No.: 18/492,486

(22) Filed: Oct. 23, 2023

(51) Int. Cl. A61K 8/46; A61K 8/19; A61K 8/22; A61K 8/41; A61Q 5/04

(52) U.S. Cl. CPC A61K 8/46; A61K 8/19; A61K 8/22; A61K 8/41; A61Q 5/04; A61K 2800/88

(57) ABSTRACT

Disclosed is the use of certain alkanolamines for use in hair treatment compositions as a replacement for some or all ammonium hydroxide. These compositions are useful for curling and straightening applications. Benefits include similar or reduced hair damage; relatively mild alkalinity; very good curl or straightening efficiency and shape retention; no heating required; and useful results were achieved with different hair ethnicities.

### FIELD OF THE INVENTION

The present invention is in the field of hair treatment applications. Specifically, it is concerned with alternatives to ammonium hydroxide for use in alkaline curling and straightening applications.

### BACKGROUND

Human hair fiber has an outermost layer, called the cuticle. The cuticle comprises about 2-12 layers of overlapping, flattened keratinocytes that are imbricated, or arranged in a “fish scale” arrangement in the longitudinal direction of the hair fiber. A second layer of hair fiber, below the cuticle, is the cortex. The cells of the cortex form a matrix that supports keratin protein structures. In the cortex, protein filaments made of long keratin chains are the main structural component of hair. These keratin chains are rich in the sulfur-containing amino acid, cysteine, which forms permanent, thermally stable crosslinking in the form of disulfide bonds (cystine) that form bridges between keratin chains. Human hair is approximately 14-20% cysteine. The extensive disulfide bonding of cysteine accounts for approximately one-third of the hair's strength, and also makes hair generally insoluble, except in specific dissociating or reducing agents.

The natural shape and structural integrity of human hair fiber depend, in part, on the orientation of the disulfide bonds which link the protein chains. They also depend on the secondary structure of the keratin fibers. However, it is generally understood that alteration of the disulfide bonds is necessary and/or useful to effect long term changes in the shape of human hair, so that treatments that do not rearrange the disulfide bonds may result only in temporary changes in hair shape or are less effective at long term reshaping of hair. For example, the use of heat and moisture to style hair may create temporary waving of the hair. However, the styled hair will return to its natural shape after a short time, as a result of exposure to moisture in the air or washing. The use of heat and moisture to style hair may break and reconfigure hydrogen bonds in the hair, but the disulfide bonds are not substantially affected. It is understood that hydrogen bonds, by themselves, are insufficient to hold the shape of hair for a significant time, because the stronger disulfide bonds eventually force the hair to reassume its original shape.

Throughout this description, “hair perming” and “permanent waving” refer to curling or straightening hair by cleaving and reforming a substantial number of disulfide bonds. Various types of permanent wave products are known. The oldest are true acid perms and alkaline perms. More recent types of perming products include acid balance waves, exothermic waves, endothermic waves, ammonia free waves, thio-free waves and low pH waves.

### Hair Types

The effectiveness of any one hair perming treatment depends upon the type of hair being treated. Treatments will vary depending on the exact type of hair. For purposes of hair styling, the Andre Walker Hair Typing System is well known in the art for classifying hair types. A modified version utilizes a twelve-fold classification system, wherein each of four main hair types are further divided into three sub-types, A, B and C (to indicate the degree of the main type, from less to more). A description of the four main hair types follows.
Type 1 (Straight hair): Straight hair types tend to be thin and flat, with little to no volume. Type I hair has more layers of cuticles, making it the most resistant hair type to conventional permanent waving treatments.
Type 2 (Wavy hair): Type 2 hair lies in between type 1 (straight) and type 3 (curly) hair. Type 2 hair can be styled either curly or straight.
Type 3 (Curly hair): Type 3 hair is characterized by curls that range from loose to springy ringlets or spirals to tight corkscrews. Type 3C hair is characterized by tight corkscrews and high volume with less smoothness than 3A and 3B hair.
Type 4 (Kinky Hair): Type 4 hair comprises tightly coiled locks. Type 4 hair is the most dry and fragile, and therefore, traditional chemical and heat styling is often avoided.

### Hair Perming Methods

Hair perming (i.e. straightening and curling) by treating the hair with chemical agents is well known. A typical process generally involves the steps of “sectioning” and “shaping” the hair, followed by the treatment steps of “softening,” “molding,” and “fixing.”

### Sectioning and Shaping

Typically, the hair to be treated is divided into sections. Then, mechanical stress is applied to each section of the hair by placing the hair in a desired shape and holding that shape with clips, curlers and/or other devices. For example, if the desired treatment is curling hair, then sections of the hair may be wound around relatively small curlers. Some of the well-known configurations include single wind perms such as, the basic, brickwork, spiral, directional and root lift perms, and double wind perms, such as the piggyback and hopscotch perms. If the desired treatment is straightening hair, then sections of hair are maintained in a straight or at least less curly configuration with clips and/or large curlers and/or other devices.

### Softening and Molding

Before a substantial number of disulfide bonds can be cleaved and reformed, the cuticle must be penetrated. Each section of hair is saturated with a lotion that contains an alkalizing agent that is able to soften and swell the cuticle sufficiently, to allow certain reducing species to reach the cortex. In various types of hair treatments where softening and swelling of the cuticle is required, ammonia (in solution) is considered the ‘gold standard’. As is commonly done, ammonia is provided as a mixture of ammonium hydroxide and ammonium thioglycolate (known as perm salt). In solution, ammonium thioglycolate is in equilibrium with free ammonia and thioglycolic acid. The free ammonia acts as an alkalizing agent, raising the pH of hair and causing the hair cuticles to soften and swell, so that actives and/or reagents can penetrate into the hair. After the cuticle has been softened, the reducing agent (i.e. thioglycolic acid) is able to cleave disulfide bonds in the cortex. When molecules of thioglycolic acid approach sufficiently close to disulphide bonds in the cortex of hair, the bonds will cleave. In the process, cystine is reduced to cysteine. For the reducing agent to be effective, it may also be necessary to include a pH adjusting agent in the lotion, although this function may also be performed by the alkalizing agent.

The use of ammonia in hair products has a number of drawbacks. For example, when in use, ammonia gas readily escapes into the ambient environment, giving off a strong malodor, as well as irritating the skin, eyes, nose and throat. Also, hair treatments with ammonium hydroxide result in some level of damage to the hair fiber. These adverse effects may be experienced by the person whose hair is being treated, as well as by the person providing the treatment.

For these reasons, research into alternative cuticle penetration methods has been ongoing for several decades, with mixed results. For example, because of their low odor, aminomethyl propanol (AMP) and monoethanolamine (MEA) have been used as replacements for ammonium hydroxide in some applications. Both molecules are known to be used in cosmetic formulations as a pH adjuster. In terms of their action on hair, the amine functional group, —NH₂, reacts similarly to ammonia (NH₃) in ammonium thioglycolate solution, while significantly reducing the ammonia odor. Nevertheless, a substantial increase in hair fiber damage has been associated with AMP and MEA, and this remains a major concern in the field.

Common perming solutions can be broadly separated into alkaline perms and acid perms. Common reducing lotions include solutions of sodium thioglycolate (pH=9-11), and ammonium thioglycolate (pH=8.5-9.5) for alkaline perms, and solutions of glyceryl monothioglycolate (pH=6.5-7.0) for acid perms. Acid perms tend to be gentler on the hair, because glyceryl monothioglycolate tends to break fewer disulfide bonds than the alkaline perming agents.

### Fixing

Following the molding stage, the hair may typically be thoroughly rinsed to remove as much reducing agent as possible. Next, an oxidizing agent (such as hydrogen peroxide or sodium bromate) that is able to neutralize the effects of the reducing agent is applied, and allowed to dwell on the hair for a short time, typically 5-10 minutes. This allows the formation of new disulfide bonds, and fixes the hair in its new shape. Thereafter, the hair is rinsed with water to remove the neutralizer. Optionally, an antioxidant conditioner may be applied to the hair to stop any residual oxidation process, bring the pH of the hair closer to a neutral range, and smooth and seal the cuticle layer. Depending on the type and condition of the hair being treated, additional steps may be needed anywhere in the process or as pre-or post-treatment steps.

The concerns of using ammonium hydroxide, AMP or MEA in hair treatment were noted above. Some of these concerns have been addressed in co-owned application U.S. Ser. No. 17/656,998, which discloses the use of certain alkanolamines in hair coloring compositions as a replacement for some or all of the ammonium hydroxide. When used as hair colorant alkalizers, these alkanolamines demonstrated reduced hair fiber damage, effective color lifting, and significant improvement in cytotoxicity and malodor compared to well-known replacements for ammonium hydroxide, AMP and MEA. The description that follows will demonstrate the usefulness of certain alkanolamines in hair perming solutions and lotions for hair styling applications.

### SUMMARY

The present application is concerned with alternatives to ammonium hydroxide for use in alkaline hair perming products, a main component of which is thioglycolic acid. Compositions of the invention also comprise certain alkanolamines that feature electron donors/acceptors, making them useful as keratin-compatible alkalizing agents for softening and swelling the cuticle of the hair. Hair perming products according to the present invention have a relatively mild pH.

### DESCRIPTION OF THE FIGURES

FIG. 1 is a scatter plot of curl efficiency verses change in denaturation temperature for Type 1 Caucasian hair subjected to hair curling treatments as described herein.

FIG. 2 is a scatter plot of curl efficiency verses change in denaturation temperature for Type 1 Chinese hair subjected to hair curling treatments as described herein.

FIG. 3 is a scatter plot of straightening efficiency versus change in denaturation temperature for Type 2C Caucasian hair subjected to hair straightening treatments as described herein.

### DETAILED DESCRIPTION

Except where otherwise explicitly indicated, all concentrations of materials and conditions of reaction, are to be understood as modified by the word “about.”

The term “comprise,” and cognates thereof, mean that a list of elements may not be limited to those explicitly recited.

Specific examples set forth herein are illustrative only, and the present invention is not limited to those mentioned examples.

### Alkalizing Agents

It can be shown that certain C3-C5 alkanolamines are useful as alkalizing agents in hair perming applications, either alone or in combination. Alkanolamines are comprised of an alkane backbone that has amino and hydroxyl functional groups. These relatively large, organic molecules are not as volatile as ammonia. However, like ammonia, alkanolamines, in general, are able to create a strongly basic environment that is potentially damaging to hair and skin cells. It is generally thought that the amine group is responsible for damage to the hair. In fact, depending on the concentration required to reproduce the benefits of ammonia in hair perming applications, some alkanolamines may produce more odor and/or damage than ammonia. We tested the seven alkanolamine alkalizing agents shown in Table 1 to determine if any of them offer performance benefits over ammonia, aminomethyl propanol (AMP) and monoethanolamine (MEA). (Note, ammonium hydroxide, MEA and AMP are included in Table 1 for comparison only).

TABLE 1
Alkalizing Agents

| Alkalizer | Molecular | Structural formula for | Molecular Weight for | |
|---|---|---|---|---|
| (INCI name) | formula | active component | active component | pKa |
| Ammonium hydroxide | NH₄OH | 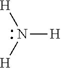 | 17.03 | 9.24 |
| Monoethanol- amine (MEA) | C₂H₇NO | 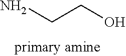 | 61.08 | 9.50 |
| Aminomethyl propanol (AMP) | C₄H₁₁NO | 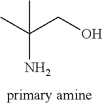 | 89.13 | 9.82 |
| DL-2-amino-1- propanol (DL-2-AP) | C₃H₈NO | 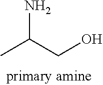 | 75 | 9.6 |
| 2-Dimethyl- amino-2-methyl- 1-propanol (DMAMP) | C₆H₁₅NO | 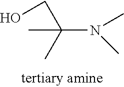 | 117.19 | 10.2 |
| 2-Amino-2- methyl-1,3- propanediol (AMPD) | C₄H₁₁NO₂ | 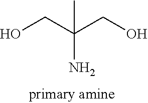 | 105.14 | 8.80 |
| 2-Amino-2- ethyl-1,3- propanediol (AEPD) | C₅H₁₃NO₂ | 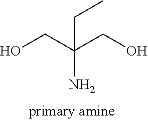 | 119.14 | 8.80 |
| 3-Amino-1,2- propanediol (Isoserinol) | C₃H₉NO₂ | 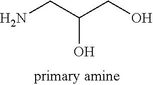 | 91.11 | — |
| 2-Amino-1,3- propanediol (Serinol) | C₃H₉NO₂ | 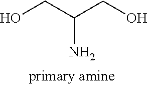 | 91.11 | — |
| Tromethamine (a.k.a Tris) | C₄H₁₁NO₃ | 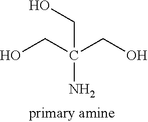 | 121.14 | 8.1 |

In determining which of these compounds or combinations thereof may offer performance benefits over ammonia, aminomethyl propanol (AMP) and monoethanolamine (MEA), a study was made of the ability of each compound to curl and straighten hair, and the degree of damage caused by applying the compounds to the hair. These results are discussed below.

### Hair Perming Product

In practice, a hair perming product or kit consists of two containers. The first container (I) contains a hair perming composition that comprises a cuticle softening agent and a disulfide bond reducing agent. Hair perming compositions are typically packaged in air tight plastic bottles, pouches, tubes or the like. The second container (II) contains a neutralizing agent composition. The contents of containers I and II are applied sequentially to the hair as explained above.

### I. The Hair Perming Composition

Hair perming compositions of the invention comprise an aqueous solution of one or more alkanolamine alkalizing agents and one or more thiol reducing agents. The alkanolamine alkalizing agents are effective to soften and swell the hair cuticle, while the thiol reducing agents are able to cleave disulfide bonds in the hair. The alkanolamine alkalizing agents of Table 1 are able to interact with thioglycolic acid reducing agent to form a salt. In solution, alkanolamine-thioglycolate salt exists as in equilibrium mixture with free thioglycolic acid and alkanolamine. In the hair perming compositions of the present invention, the concentration of thioglycolic acid is typically at about 5% to 8% by weight of the hair perming composition. Also, the molar ratio of alkalizer(s) to thioglycolic acid should be at least 1, preferably at least 1.25, more preferably at least 1.50, even more preferably at least 1.75 to ensure that the pH of the hair perming composition is greater than 7, and preferably at least 8.

Optionally, various auxiliary ingredients may be included which impart a benefit to the perming composition or to the hair. For example, hair benefit ingredients may include conditioners, natural oils, amino acids, peptides, proteins, vitamins, chelators, antioxidants, etc.

### II. The Neutralizing Agent Composition

A neutralizing agent composition for use in the present invention comprises an oxidizer, typically hydrogen peroxide. This allows the formation of new disulfide bonds, and fixes the hair in its new shape. Preferably, the hydrogen peroxide neutralizer is at least at 1% (w/w). Other neutralizers such as sodium bromate or sodium perborate etc. could also be used.

### Hair Perming Compositions Tested

For testing, we prepared the hair perming compositions shown by Tables 2 and 3, below. Table 2 shows a base hair perming composition (without the alkalizer agent). Table 3 shows the amount of each individual alkalizer that was added to one composition of Table 2 to complete a hair perming composition, as well as the pH of each hair perming composition. All compositions contain the same molar percentage of alkalizer, the water content being adjusted accordingly.

TABLE 2
Base Perming Composition

| INCI Name | Percent |
|---|---|
| Thioglycolic acid (>=97%) | 7.50 |
| Glycerin | 2.00 |
| Polysorbate 20 | 1.00 |
| Tetrasodium EDTA | 0.40 |
| Water | q.s. |

TABLE 3

| | | |
|---|---|---|
| | Percent (by weight | |
| | of total alkalizer | |
| Alkalizer | composition) | pH |

Comparative Examples

| | | |
|---|---|---|
| Ammonium hydroxide (29%)* | 8.20 | 9.14 |
| MEA (99%)* | 8.61 | 9.40 |
| AMP (98%)* | 12.82 | 9.63 |
| NaOH (50%) | 11.28 | 10.56 |

Alkalizer Test Samples

| | | |
|---|---|---|
| DL-2-AP | 10.58 | 9.45 |
| DMAMP (78%)* | 21.18 | 10.18 |
| AMPD | 14.82 | 9.04 |
| AEPD (97%)* | 17.32 | 9.09 |
| Isoserinol | 12.85 | 9.18 |
| Serinol (97%)* | 13.24 | 8.68 |
| Tris | 17.08 | 8.36 |

*percent active

Ammonium hydroxide, which has been the gold standard in alkalizers, serves as a control, and MEA and AMP as common replacements for ammonium hydroxide are included for comparison. Also included, for comparison purposes, are hair samples treated with NaOH, which, above a certain concentration, is a very potent alkalizer that induces significant damage in human hair. Therefore, NaOH is included as a worst damage level indicator. All hair perming compositions were tested for curl efficiency, straightening efficiency and keratin protein damage. Data are also analyzed to evaluate the effect of pH on the performance of the perming compositions.

### Curl Efficiency, Straightening Efficiency

The ability of each test composition to curl hair was established by measuring “curl efficiency”. Curl efficiency is a measure of curl tightness following a hair curling treatment. The higher the efficiency, the tighter the curl. We used a well-known method of measuring curl efficiency, wherein a portion of untreated straight hair of known length, Lu, is secured on an arrangement of pegs to form a sine wave pattern of five cycles, crest-to-crest. Because the pegs are fixed, the ideal length, Li, of the portion of hair when arranged into the five-cycle wave pattern, is known. Starting with a straight swatch of hair of length Lu, the ideal outcome would be a curled swatch of hair with a length of Li. Curl efficiency is a measure of how closely the ideal is attained.

The ability of each test composition to straighten curly hair was established by measuring “straightening efficiency”. Straightening efficiency represents the degree to which curly hair is made straight by a hair straightening treatment. We start with a curly, untreated hair tress of length Lu. The tress has an ideal straight length of Li, which is the length of the untreated tress when flat ironed to its maximum, straight length. After treatment the tress has a length of Lt. Straightening efficiency is the gain in length as a percentage of the maximum possible gain in length. A greater straightening efficiency indicates a superior straightening effect. A straightening efficiency of 100% means that the treated tress retained the maximum possible length.

### Test Sample Preparation

Two types of hair samples were tested for curling; mixed sourced virgin Type 1Caucasian hair, and single sourced virgin Type 1 Chinese hair. For straightening, mixed sourced virgin Type 2C (very wavy) Caucasian hair was tested. Hair straightening test samples were prepared by collecting hair fibers into tresses, and clamping each tress at both ends. For those test samples that were to undergo a straightening treatment, the length, Lu, was measured between two previously determined points.

### Curling/Straightening Stage

Each hair tress was wetted (reverse osmosis water). For curling, each hair tress was wound along a peg board to form a sine wave pattern of five cycles, as described above. For straightening, each hair tress was held on a board in a straight conformation, tension was applied, and the ends of the tress were secured. All test samples (curling or straightening) were allowed to dry before proceeding. Each board with a hair tress test sample was dipped for 10 seconds into a beaker with one of the hair perming compositions. After removing a board from solution, it was immediately placed in an oven for 20 minutes at 37° C. (normal body temperature). The test sample was removed from the oven, and rinsed for five minutes with 37° C. water, being careful not to allow the running water to fall directly on the hair tresses. After blotting with a paper towel, test samples were allowed to rest for five minutes at room temperature, for air oxidation to occur. The hair straightening test samples were removed from the board, blow dried and flat ironed at 200° C., which is commonly done in salon practice. At this point, the ideal length, Lᵢ, was measured between the two previously determined point mentioned above. Thereafter, each sample was rebound to the board.

### Fixing Stage

Each bound hair tress was submerged for ten seconds in a 2% solution of H₂O₂ to neutralize the reducing agent and promote S-S bond formation, so that the hair tress would retain a desired shape (sine wave pattern or straight) when removed from the board. Each hair tress rested at room temperature for about five minutes, then was rinsed under 37° C. running water for five minutes, and subsequently blotted with a paper towel. Each test sample hair tress was removed from the board, laid on a flat surface, and allowed to dry. The fully permed dry tresses were washed in 5% SDS (sodium dodecyl sulfate) solution (which simulates salon practice) and allowed to dry overnight.

### Additional Measurements

For the curling test samples, the length, Lt, of five waves, crest to crest was measured. For the straightening test samples, the final length, Lt, was measured between the two previously determined points. At this point, differential scanning calorimetry (DSC) measurements were made on a portion of each hair tress test sample. Also, for longevity performance, each tress was subjected to the equivalent of eight hand washings in 5% SDS solution using the GyroWash color fastness to laundering tester, and allowed to dry, before another length measurement, Lₜ, was made.

### Calculations

Curl efficiencies were computed using Equation 1.

$$\% Curl Efficiency = [ ( {L}_{u} - {L}_{t} ) / ( {L}_{u} - {L}_{i} ) ] * 100 \qquad ( Equation 1 )$$

Lᵤ=length of the untreated hair tress
Lₜ=crest-to-crest length of the five-wave portion of the treated hair tress
Lᵢ=ideal length of curled hair

(Lᵤ−Lᵢ) represents the maximum possible reduction in length as a result of treatment. (Lᵤ−Lₜ) represents the actual reduction of length as a result of treatment. Curl efficiency represents the degree to which a curl does not revert to its original length (straighten) when removed from the pegs.

Straightening efficiencies were computed using Equation 2.

$$\% Straightening Efficiency = [ ( {L}_{t} - {L}_{u} ) / ( {L}_{i} - {L}_{u} ) ] ] * 100 \qquad ( Equation 2 )$$

Lᵤ=length of the untreated hair tress
Lₜ=length of treated hair tress
Lᵢ=ideal length of straightened hair

(Lᵢ−Lᵤ) represents the maximum possible increase in length as a result of treatment. (Lₜ−Lᵤ) is the actual increase in length as a result of treatment. Straightening efficiency represents the degree to which straightened hair does not revert to its original length (curl up) when tension is removed.

### RESULTS AND DISCUSSION

Curl efficiency results for Type 1 Caucasian hair and Type 1 Chinese hair are shown in Tables 4 and 5, respectively. The left column is the alkalizer in the base perming composition (Table 2) used to treat the hair. Second from left is the curl efficiency of the treated samples after being washed once with a 5% SDS solution and allowed to dry. Second from the right is the curl efficiency of the same sample after the equivalent of 8 washes with a 5% SDS solution and allowed to dry. The right-most column shows the percent change in curl efficiency between 1 wash and 8 washes, which indicates how well or poorly the treated hair retained curl after eight washes.

TABLE 4
Type 1 Caucasian Hair

| | | | |
|---|---|---|---|
| | Curl | Curl | |
| | Efficiency | Efficiency | % Change |
| | (%) After | (%) After | In Curl |
| | Treatment, | Treatment, | Efficiency |
| | 1 Wash and | 8 Washes | From 1 to 8 |
| Alkalizer | Dry | and Dry | Washes |

Comparative Examples

| | | | |
|---|---|---|---|
| Ammonium hydroxide | 51.40 | 46.73 | −9.09 |
| MEA | 50.47 | 46.73 | −7.41 |
| AMP | 49.53 | 45.79 | −7.55 |
| NaOH | 47.66 | 39.25 | −17.64 |

Alkalizer Test Samples

| | | | |
|---|---|---|---|
| DL-2-AP | 50.47 | 44.86 | −11.11 |
| Isoserinol | 48.60 | 44.86 | −7.69 |
| Serinol | 45.79 | 42.06 | −8.15 |
| DMAMP | 44.86 | 35.48 | −20.91 |
| AEPD | 43.93 | 41.12 | −6.40 |
| AMPD | 42.99 | 37.38 | −13.05 |
| Tris | 38.32 | 36.45 | −4.88 |

TABLE 5
Type 1 Chinese Hair

| | | | |
|---|---|---|---|
| | Curl | Curl | |
| | Efficiency | Efficiency | % Change |
| | (%) After | (%) After | In Curl |
| | Treatment, | Treatment, 8 | Efficiency |
| | 1 Wash and | Washes and | From 1 to 8 |
| Alkalizer | Dry | Dry | Washes |

Comparative Examples

| | | | |
|---|---|---|---|
| Ammonium hydroxide | 61.68 | 53.27 | −13.63 |
| MEA | 64.77 | 56.07 | −13.43 |
| AMP | 61.03 | 54.21 | −11.17 |
| NaOH | 54.86 | 43.93 | −19.92 |

Alkalizer Test Samples

| | | | |
|---|---|---|---|
| DL-2-AP | 69.81 | 55.14 | −21.01 |
| Isoserinol | 59.81 | 53.27 | −10.93 |
| DMAMP | 58.60 | 48.60 | −17.06 |
| AEPD | 53.93 | 45.79 | −15.09 |
| AMPD | 52.34 | 48.60 | −7.15 |
| Serinol | 50.47 | 42.99 | −14.82 |
| Tris | 37.38 | 33.64 | −10.01 |

All seven of the tested alkalizers are effective at imparting curl to Type 1 Caucasian and Type 1 Chinese hair, and all samples retained a substantial amount of curl (about 33% to 55% curl efficiency) after the equivalent of eight washes. On both Type 1 Caucasian and Type 1 Chinese hair, DL-2-AP is best at imparting curl, either similar to or even better than the gold standard, ammonium hydroxide, as well as AMP and MEA, the common replacements for ammonium hydroxide. Of the seven alkalizers tested, the next best at imparting curl to Caucasian and Chinese hair was Isoserinol. On both Caucasian and Chinese hair, after the equivalent of 8 washes, the samples treated with DL-2-AP still had the most curl, although, as a percentage of initial curl, these samples lost the most curl of the tested samples. After the equivalent of 8 washes, the samples treated with Isoserinol ranked second for curl, while the amount of curl lost was comparable to or better than the standards, ammonium hydroxide, AMP and MEA. All of the tested alkalizing agents were useful for curling hair, a substantial amount of which was retained, even after the equivalent of eight washes.

Straightening efficiency results for Type 2C Caucasian hair are shown in Table 6. The left column is the alkalizer in the base perming composition (Table 2) used to treat the hair. The right column is the straightening efficiency of the treated samples after being washed once with a 5% SDS solution and allowed to dry.

TABLE 6
Type 2C Caucasian Hair

| | |
|---|---|
| | Straightening |
| | Efficiency (%) |
| | After Treatment, |
| Alkalizer | 1 Wash and Dry |

Comparative Examples

| | |
|---|---|
| Ammonium hydroxide | 75.00 |
| MEA | 65.38 |
| AMP | 85.71 |
| NaOH | 65.71 |

Alkalizer Test Samples

| | |
|---|---|
| DL-2-AP | 95.35 |
| Isoserinol | 97.30 |
| DMAMP | 93.75 |
| AEPD | 94.70 |
| AMPD | 77.42 |
| Serinol | 88.24 |
| Tris | 82.50 |

All seven of the tested alkalizers were effective at straightening Type 2C Caucasian hair. Five of the seven produced significantly better results than ammonium hydroxide, AMP and MEA. AMPD and Tris also performed better than ammonium hydroxide and MEA, but not as well as AMP. Nevertheless, all of the tested alkalizing agents were useful for straightening hair.

### DSC Analysis

As noted above, hair perming treatments cause some level of damage to the hair fiber. This damage occurs at the level of keratin proteins. Damage in the form of protein denaturation occurs when proteins lose some of their secondary, tertiary or quaternary structure by application of some external stress or compound, such as a strong acid or base, a concentrated inorganic salt, an organic solvent (e.g., alcohol or chloroform) or heat. It is known that the denaturation of keratin in hair can be detected by differential scanning calorimetry (DSC). DSC is a thermal analysis technique used to measure phase transition temperatures and heat of transformation (enthalpy) for endothermic and exothermic reactions. DSC is sensitive enough to provide information about molecular weight distributions of polymers.

For all of the test samples discussed above, DSC measurements were performed using the Discovery DSC 2500 (from TA Instruments. New Castle, DE). The experiments were carried out over a temperature range of 40° C. to 200° C., with a scan rate of 5° C./min under nitrogen protection. DSC samples were prepared by cutting tress samples into pieces (0.1 to 1.0 mm in size) and weighing. The hair samples were mixed with deionized water, and then sealed in high volume pans for at least 6 hours before measuring. The phase transition temperature (keratin denaturation temperature) of each hair sample was analyzed using TRIOS software (Discovery DSC 2500). Untreated hair was used as a control.

Each hair sample was analyzed at least twice, and the average temperature was obtained for data analysis. This was subtracted from the denaturation temperature of the control sample (ΔT=T_{Untreated}−T_{Alkalizer}). A larger ΔT indicates more damage. For Type 1 Caucasian hair subjected to a curling treatment, DSC results are shown in Table 7, which is arranged from greatest ΔT (most damage) to lowest (least damage).

TABLE 7
Type 1 Caucasian Hair After Curling Treatment

| | | |
|---|---|---|
| | | pH of alkalizer |
| Alkalizer | ΔT (° C.) | composition |

Comparative Examples

| | | |
|---|---|---|
| NaOH | 6.34 | 10.56 |
| MEA | 4.44 | 9.40 |
| AMP | 3.11 | 9.63 |
| Ammonium hydroxide | 1.96 | 9.14 |

Alkalizer Test Samples

| | | |
|---|---|---|
| DMAMP | 6.06 | 10.18 |
| DL-2-AP | 3.95 | 9.45 |
| Isoserinol | 3.07 | 9.18 |
| AEPD | 2.36 | 9.09 |
| Serinol | 1.76 | 8.68 |
| AMPD | 1.66 | 9.09 |
| Tris | 1.11 | 8.36 |

The above results indicate that, when used in hair curling treatments on Type 1Caucasian hair, six of the seven alkalizers test samples (Tris, AMPD, Serinol, AEPD, Isoserinol, DL-2-AP) produced similar or significantly less damage to Caucasian hair than ammonium hydroxide and/or at least one of the common replacements for ammonium hydroxide, MEA and AMP. Tris, AMPD and Serinol produced less damage than ammonium hydroxide. Only DMAMP (a tertiary amine with one OH group) performed less well, causing almost as much damage as NaOH.

For Type 1 Chinese hair subject to curling treatment, DSC results are shown in Table 8, which is arranged from greatest ΔT (most damage) to lowest (least damage).

TABLE 8
Type 1 Chinese Hair After Curling Treatment

| | | |
|---|---|---|
| | | pH of alkalizer |
| Alkalizer | ΔT (C. °) | composition |

Comparative Examples

| | | |
|---|---|---|
| NaOH | 5.60 | 10.56 |
| MEA | 1.80 | 9.40 |
| AMP | 1.65 | 9.63 |
| Ammonium hydroxide | 0.80 | 9.14 |

Alkalizer Test Samples

| | | |
|---|---|---|
| DMAMP | 5.78 | 10.18 |
| DL-2-AP | 3.49 | 9.45 |
| Isoserinol | 2.51 | 9.18 |
| AEPD | 1.31 | 9.09 |
| AMPD | 0.68 | 9.04 |
| Serinol | 0.62 | 8.68 |
| Tris | −0.77 | 8.36 |

The above results indicate that, when used in hair curling treatments on Type 1 Chinese hair, four of the seven alkalizers tested (Tris, Serinol, AMPD, AEPD) produced similar or significantly less damage to Chinese hair than ammonium hydroxide and/or either of the common replacements for ammonium hydroxide, MEA and AMP. Tris, AMPD and Serinol produced less damage than ammonium hydroxide. Furthermore, while, in this test, Isoserinol and DL-2-AP did not perform as well as the other alkalizers, nor as well as ammonium hydroxide, MEA or AMP, the absolute level of damage incurred suggests that Isoserinol and DL-2-AP are still useful for perming treatments. Again, the alkalizer composition with DMAMP (a tertiary amine with one OH group) caused the most damage, even worse than NaOH.

For Type 3 Caucasian hair subject to straightening treatment, DSC results are shown in Table 9, which is arranged from greatest ΔT (most damage) to lowest (least damage).

TABLE 9
Type 3 Caucasian Hair After Straightening

| | | |
|---|---|---|
| | | pH of alkalizer |
| Alkalizer | ΔT (° C.) | composition |

Comparative Examples

| | | |
|---|---|---|
| NaOH | 4.60 | 10.56 |
| AMP | 2.60 | 9.63 |
| MEA | 2.32 | 9.40 |
| Ammonium hydroxide | 0.66 | 9.14 |

Alkalizer Samples

| | | |
|---|---|---|
| DL-2-AP | 3.20 | 9.45 |
| Isoserinol | 2.17 | 9.18 |
| DMAMP | 1.43 | 10.18 |
| AEPD | 1.13 | 8.98 |
| Serinol | 0.50 | 8.68 |
| AMPD | 0.32 | 9.04 |
| Tris | 0.07 | 8.36 |

The above results indicate that, when used in hair straightening treatments on Type 3Caucasian hair, six of the seven alkalizers tested produced similar or significantly less damage to Caucasian hair than ammonium hydroxide and/or either of the common replacements for ammonium hydroxide, MEA and AMP. Tris, AMPD and Serinol produced less damage than ammonium hydroxide. DL-2-AP used in straightening treatment produced more damage than ammonium hydroxide, MEA or AMP. Unlike the curling treatments, DMAMP, produced comparatively little damage to Caucasian hair during straightening.

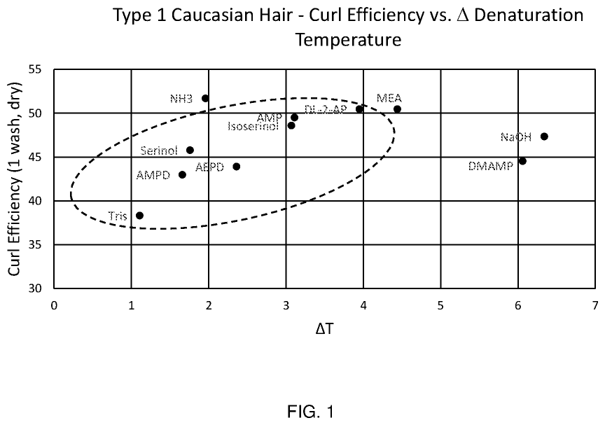

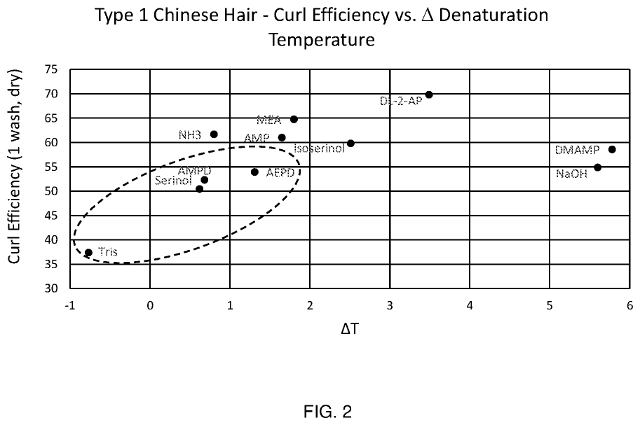

FIGS. 1, 2 and 3 provide visual presentations of the results. In FIGS. 1 and 2, those alkalizer test samples located inside the oval compare well with the ammonium hydroxide, AMP and MEA test samples, in curling, curl retention, and degree of damage. In terms of curling treatment, only DMAMP (a C6 alkanolamine) did not perform as well as ammonium hydroxide, AMP or MEA. DL-2-AP, AMPD, AEPD, Isoserinol, Serinol, Tris are C3-C5 alkanolamines.

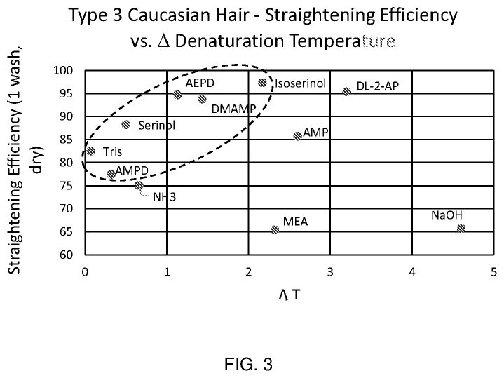

In FIG. 3, alkalizer test samples located inside the oval compare well with the ammonium hydroxide, AMP and MEA test samples in both straightening and degree of damage. In terms of straightening, DMAMP did perform well, while DL-2-AP did not.

We have demonstrated that hair perming compositions formed from thioglycolic acid and certain C3-C5 alkanolamines, especially those with two or more hydroxyl groups, are useful in hair perming applications (curling and straightening). Thioglycolic acid plus selected alkanolamines offer superior curling or straightening efficiency, excellent shape retention (even through the equivalent of 8 washes), and cause less hair damage than the gold standard ammonium hydroxide, or MEA and AMP. The hair perming compositions are useful for hair of different ethnicities, and have a relatively mild alkalinity (pH of about 8.3 to 9.5), making them suitable for commercial hair care products. The least consistent results were achieved with DMAMP and DL-2-AP, both having only one hydroxyl group. But even these demonstrated some usefulness.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair perming composition that comprises:
5% to 8% of thioglycolic acid by weight of the hair perming composition; and
one or more C3-C5 alkanolamines in a molar ratio to thioglycolic acid of at least 1;
wherein the pH of the hair perming composition is greater than 7.

2. The hair perming composition of claim 1 wherein the one or more alkanolamines are selected from DL-2-amino-1-propanol (DL-2-AP), 2-Amino-2-methyl-1,3-propanediol (AMPD), 2-Amino-2-ethyl-1,3-propanediol (AEPD), 3-Amino-1,2-propanediol (Isoserinol), 2-Amino-1,3-propanediol (Serinol), Tromethamine (Tris) and 2-Dimethyl-amino-2-methyl-1-propanol (DMAMP).

3. The hair perming composition of claim 2 wherein the molar ratio of the one or more C3-C5 alkanolamines to thioglycolic acid is at least 1.0.

4. The hair perming composition of claim 2 wherein the pH of the hair perming composition is at least 7.0.

5. A hair perming product comprising a first container and a second container, wherein:
the first container contains a hair perming composition according to claim 2; and
the second container contains a neutralizing agent composition that comprises hydrogen peroxide, sodium bromate or a combination thereof.

6. The hair perming composition of claim 5 wherein the molar ratio of the one or more C3-C5 alkanolamines to thioglycolic acid is at least 1.0.

7. The hair perming product of claim 5, wherein said hair perming composition has a pH from 7.0 to 10.6

8. A method of curling hair comprising the steps of applying a hair perming composition according to claim 2 to Type 1 Caucasian hair.

9. A method of curling hair comprising the steps of applying a hair perming composition according to claim 2 to Type 1 Chinese hair wherein the one or more alkanolamines are selected from 2-Amino-2-methyl-1,3-propanediol (AMPD), 2-Amino-2-ethyl-1,3-propanediol (AEPD), 3-Amino-1,2-propanediol (Isoserinol), 2-Amino-1,3-propanediol (Serinol), and Tromethamine (Tris).

10. The method of claim 9 wherein the one or more alkanolamines are selected from 2-Amino-2-methyl-1,3-propanediol (AMPD), 2-Amino-2-ethyl-1,3-propanediol (AEPD), 2-Amino-1,3-propanediol (Serinol), and Tromethamine (Tris).

11. A method of straightening hair comprising the steps of applying a hair perming composition according to claim 2 to Type 2C Caucasian hair wherein the one or more alkanolamines are selected from 2-Amino-2-methyl-1,3-propanediol (AMPD), 2-Amino-2-ethyl-1,3-propanediol (AEPD), 3-Amino-1,2-propanediol (Isoserinol), 2-Amino-1,3-propanediol (Serinol), Tromethamine (Tris) and 2-Dimethyl-amino-2-methyl-1-propanol (DMAMP).
